(19) United States
(12) Patent Application Publication
(10) Pub. No.: US 2009/0258373 A1
(43) Pub. Date: Oct. 15, 2009

(54) METHODS OF CONTROLLING THE SENSITIVITY AND DYNAMIC RANGE OF A HOMOGENEOUS ASSAY

(75) Inventors: Kristin Weidemaier, Raleigh, NC (US); W. Shannon Dillmore, Raleigh, NC (US)

Correspondence Address: David W. Highet, VP & Chief IP Counsel;Becton, Dickinson and Company, (Lerner David Littenberg), 1 Becton Drive , MC 110, Franklin Lakes, NJ, 07417-1880, US

(73) Assignee: BECTON, DICKINSON AND COMPANY, Franklin Lakes, NJ (US)

(21) Appl. No.: 12/422,381

(22) Filed: Apr. 13, 2009

Related U.S. Application Data
(60) Provisional application No. 61/044,081, filed on Apr. 11, 2008.

(51) Int. Cl. G01N 33/53; G01N 33/566

(52) U.S. Cl. 435/7.1; 436/501

(57) ABSTRACT

A method is disclosed for accurately determining the concentration of a target analyte utilizes reagent pairs having different affinity for the target. The different affinity provides distinct binding profiles that can be analyzed to absolutely determine the analyte concentration. The method provides an assay system having expanded dynamic range to cover a wider range of analyte concentration and can overcome the hook-effect that commonly exists in homogenous assay systems. The method utilizes distinguishable signals that allows for the analysis of multiple binding profiles and multiplex analysis.

### CROSS-REFERENCE TO RELATED APPLICATIONS

This application claims the benefit of the filing date of U.S. Provisional Patent Application No. 61/044,081 filed Apr. 11, 2008, the disclosure of which is hereby incorporated herein by reference.

### FIELD OF THE INVENTION

The present invention relates to a novel method for determining the concentration of a target analyte utilizing an affinity binding assay. In particular, the present invention relates to an assay having improved sensitivity and an expanded dynamic range that can overcome the “hook-effect.”

### BACKGROUND

The quantitative determination of substances by means of affinity binding assays is known. In particular, the determination of antigenic substances by means of immunoassays is known. In conventional immunoassays, detection of an antigen can occur by “sandwiching” the antigen between two antibodies, a detection antibody which is labeled with an optical or calorimetric reporter, and a capture antibody which is typically coupled to a solid support. The measured signal can then be used to determine the concentration of the antigen present in the sample. A sandwich immunoassay is further illustrated in FIG. 1A, wherein a target analyte 10 is bound by a capture antibody 12 immobilized to a solid support 14, and bound by a detection antibody 16 labeled with a signal particle 18. Conventional Enzyme Linked Immunosorbent Assay (ELISA) is an example of this type of technology.

In conventional immunoassays, multiple wash steps may be employed to permit the detection of an analyte with high sensitivity and dynamic range. In one configuration, for example, an ELISA is performed by immobilizing a capture antibody to a solid surface. A sample containing the target analyte (e.g. a protein) is then added. Analyte that does not complex with the immobilized capture antibody is washed away, while analyte that has been bound to the capture antibody is then detected with the labeled detection antibody.

Homogeneous assays offer the potential for a significantly streamlined work flow. In homogeneous assays, wash steps are omitted, and the target is detected with minimal sample manipulation. One example of a homogeneous assay is shown in FIG. 2. In FIG. 2, a sample 30 is incubated with a capture antibody 32 immobilized to a magnetic particle 34, and a detection antibody 36 labeled with a reporter molecule 38, such as a fluorescence or Surface Enhanced Raman Spectroscopy (SERS) tag. After formation of a sandwich complex 39, a magnetic field can be applied to pull the magnetic particle complexes to a predefined region of a tube 40. The resulting pellet 42 can then be interrogated optically without the need for wash steps.

One of the inherent issues with a homogeneous assay is that the absence of wash steps may limit its dynamic range compared to a non-homogeneous assay. As antigen concentration becomes higher, the capture antibodies and detection antibodies each will bind antigen inhibiting the formation of the sandwich complex. This phenomenon manifests itself as a drop in signal, producing a so-called “hook-effect.” The hook-effect is further illustrated in FIGS. 3A and 3B.

Referring to FIG. 3A, the hook-effect can occur in the presence of excess antigen, wherein the formation of sandwich complexes is inhibited. The hook-effect occurs when capture antibody 12 (immobilized to solid support 14) and detection antibody 16 (labeled with signal particle 18) bind to separate target analytes 10, blocking formation of a sandwich complex. Referring to FIG. 3B, when Prostate-Specific Antigen (PSA) is present at low levels, the observed (optical) signal is substantially proportional to the amount of sandwich complex formed, and increasing levels of PSA result in more sandwich complexes and thus more signal. Above the region of the hook-effect, however, the signal actually decreases with increasing PSA level. As seen in FIG. 3B, the hook-effect occurs at a PSA concentration above about 100 ng/ml. The hook-effect limits the concentration range over which an assay is quantitative (i.e., the dynamic range). In the presence of excess antigen, the hook-effect can greatly limit the dynamic range of the assay.

A need exists for a homogeneous assay that can overcome the hook-effect, and has an expanded dynamic range of sensitivity.

### SUMMARY OF THE INVENTION

The present invention relates to a method for determining the concentration of a target analyte. The method can overcome the hook-effect and expand the dynamic range of accurate detection. The method can be utilized in a homogeneous assay system. The method incorporates binding moieties (e.g., antibodies, oligonucleotides) having different affinity for the analyte to generate different binding profiles. The binding profiles can be analyzed to absolutely determine the concentration of target analyte. In one or more embodiments, the method utilizes a sandwich assay in which a first binding moiety immobilized to a solid support (e.g. a “capture antibody,” or “capture oligonucleotide”), and a second binding moiety labeled with a first signal particle (e.g. a “detection antibody,” or “detection oligonucleotide”), can be incubated with a target analyte (e.g. an antigen, or a nucleic acid). A binding moiety can be a molecule(s) that binds, attaches, or otherwise associates with a specific molecule. The binding, attachment, or association can be chemical or physical. A specific molecule to which a specific binding member binds can be any of a variety of molecules, including, but not limited to, antigens, haptens, proteins, carbohydrates, nucleotide sequences, nucleic acids, amino acids, peptides, enzymes, and the like.

A first sandwich complex, such as illustrated for example in FIG. 1A, FIG. 1B, and FIG. 2, can form. The first and second binding moieties can each specifically bind to the target analyte, and each can have an established affinity for the target analyte.

A third binding moiety immobilized to a solid support, and a fourth binding moiety labeled with a second signal particle, can further be incubated with the target analyte. The third and fourth binding moieties can each specifically bind to the target analyte, and each can have an established affinity for the target analyte. Likewise, a second sandwich complex, such as illustrated in FIG. 1A, FIG. 1B, and FIG. 2, can form.

The method utilizes multiple sandwich complexes, which produce different binding profiles that correlate to the different affinity. The binding moieties can have differing binding affinities for the target analyte, and therefore, incubating the target analyte with two pairs of binding moieties can produce first and second sandwich complexes having different binding profiles. When two binding moiety pairs are used in an assay, two different response profiles can be observed. Target analyte levels can be determined by reading and analyzing the signals generated by the two binding profiles. As an option, more than two binding profiles can be created using two or more sandwich complexes. The first and second signal particles can each generate a detectable signal that is distinguishable, each from the other, and the first and second signals can be detected and measured. A first signal, generated from the first signal particle in the first sandwich complex, and a second signal, generated from the second signal particle in the second sandwich complex, can each be compared to a standard, or a standard reference profile. This is one way to quantify the result. However, it is possible to have only a single standard curve that is based on, e.g., the ratio of the first signal to the second signal. The standard and/or standard reference profile can comprise, for example, the ratio of the first signal to the second signal. Based on the comparison, the concentration of target analyte can be absolutely determined.

The method overcomes the consequences of a hook-effect, such as at high target analyte concentrations. In one or more embodiments, an analyte concentration can be determined by comparing a first signal to a first standard reference profile. Any potential ambiguity resulting from a possible hook-effect can be clarified by comparing a second signal to a second standard reference profile. The method can also provide a more accurate determination of the analyte concentration. Comparing both first and second signals to corresponding standard reference profiles can verify the target analyte concentration.

The present invention further relates to an assay system for detecting a target analyte. The system can overcome the hook-effect and can expand the dynamic range of an assay. The system can be utilized in a homogenous assay system. The system incorporates binding moieties that have different affinity for the target analyte and consequently produce different binding profiles. The binding profile data can be collected and analyzed to determine the concentration of target analyte.

The assay system can utilize a sandwich assay method wherein a sample containing the target analyte is incubated with a pair of binding moieties, one of which is immobilized to a solid support, and the other is labeled with a signal particle. The sample is also incubated with a second pair of likewise immobilized and labeled binding moieties, the second pair having different affinity for the target analyte than the first pair, and labeled with a different signal particle. Each pair of binding moieties can form a sandwich complex with the target analyte, such as illustrated in FIG. 1A, FIG. 1B, and FIG. 2. The different signal particles in each pair can generate different signals that can be distinguished from one another.

The assay system can include an instrument capable of detecting the different signals generated by the signal particles and an analyzer capable of analyzing the detected signals to determine the presence of and/or quantity of target analyte. The system can utilize one or more standard reference profiles prepared from known amounts of target analyte, and the analyzer can compare the detected signals generated from each sandwich complex to the one or more standard reference profiles to determine the quantity of target analyte present.

### BRIEF DESCRIPTION OF THE DRAWINGS

The accompanying drawings, which are incorporated into and constitute a part of the present invention, illustrate embodiments of the present invention, and together with the general description given above and the detailed description, serve to explain the principles of the present invention.

FIG. 1A is a diagrammatic representation of an immunoassay sandwich complex according to one or more embodiments of the present invention.

FIG. 1B is a diagrammatic representation of a nucleic acid-based sandwich complex according to one or more embodiments of the present invention.

FIG. 2 is a diagrammatic representation of a homogenous magnetic capture immunoassay according to one or more embodiments of the present invention.

FIG. 3A is a diagrammatic representation of an immunoassay hook-effect blocking the formation of a sandwich complex.

FIG. 3B is a graph illustrating a hook-effect in a prostate specific antigen (PSA) immunoassay.

FIG. 4A is a diagrammatic representation of binding moieties according to one or more embodiments of the present invention.

FIG. 4B is a diagrammatic representation of an assay that forms two sandwich complexes and a graph illustrating the binding profiles of the two sandwich complexes, according to one or more embodiments of the present invention.

FIG. 4C is a diagrammatic representation of an assay that forms two sandwich complexes and a graph illustrating the binding profiles of the two sandwich complexes, according to one or more embodiments of the present invention.

FIG. 5A is a diagrammatic representation of binding moiety pairs according to one or more embodiments of the present invention.

FIG. 5B is a bar graph illustrating the signal level from a thyroid stimulating hormone (TSH) assay using binding moiety pairs according to one or more embodiments of the present invention.

FIG. 6 illustrates a SERS spectrum measured according to one or more embodiments of the present invention.

FIG. 7 is a diagrammatic representation of several binding moiety pairs according to one or more embodiments of the present invention.

FIG. 8A is a diagrammatic representation of potential binding complexes that do not contribute to an assay signal.

FIG. 8B is a diagrammatic representation of binding moiety pairs.

FIG. 8C is a diagrammatic representation of binding moiety pairs.

FIG. 9A is a diagrammatic representation of a nucleic acid-based assay system according to one or more embodiments of the present invention.

FIG. 9B is a graph illustrating a nucleic acid-based assay binding profile utilizing binding moieties according to one or more embodiments of the present invention.

The drawings and the following detailed description provide information about the present invention including the description of specific embodiments. The detailed description serves to explain the principles of the present invention. The present invention is susceptible to modifications and alternative forms and is not limited to the particular forms disclosed. The present invention covers all modifications, equivalents, and alternatives falling within the spirit and scope of the invention as defined by the claims.

### DETAILED DESCRIPTION OF THE INVENTION

The present invention relates to a method for determining the concentration of a target analyte. The term “target analyte,” as used herein, is a substance to be detected in a test sample using the present invention. The analyte can be any substance for which there exists a naturally occurring capture reagent, or for which a capture reagent can be prepared. The target analyte can bind to one or more binding moieties in an assay. The target analyte can include a protein, a peptide, an amino acid, a carbohydrate, a hormone, a nucleic acid, a steroid, a vitamin, a cell, a drug, a bacterium, a virus, and metabolites of, or antibodies to, any of the above substances. The target analyte can comprise, for example, oncology markers, such as prostate specific antigen (PSA), alpha-fetoprotein (AFP), carcinoembryonic antigen (CEA), and cyclin-dependent kinase inhibitor 2A (p16), human papilloma virus proteins such as E6 and/or E7 proteins, influenza virus, hormones, such as thyroid stimulating hormone (TSH), and human chorionic gonadotropin (hCG), mini-chromosomal maintenance (MCM) family members, cardiac markers, creatine kinase-subtype MB (CK-MB) and troponin.

The term “binding moiety,” as used herein, refers to a molecule that binds, attaches, or otherwise associates with a specific molecule. The binding, attachment, or association can be chemical or physical. A specific molecule to which a specific binding member binds can be any of a variety of molecules, including, but not limited to, antigens, haptens, proteins, carbohydrates, nucleotide sequences, nucleic acids, amino acids, peptides, enzymes, and the like.

The present invention further relates to a method that can expand the dynamic range and the sensitivity of an assay. In particular, the method can expand the dynamic range and sensitivity of a homogeneous assay. As used herein, the term “homogeneous assay” refers to an assay in which no wash steps are performed to remove excess reagent or target. In particular, the present invention pertains to assays in which a target analyte is detected when it forms a sandwich complex with a capture reagent and a detection reagent.

The term “capture reagent,” as used herein, is a molecule or compound capable of binding the target analyte, which can be directly or indirectly attached to a substantially solid support. The term “detection reagent,” as used herein, refers to an agent that is capable of generating a detectable signal, which can be used to assess the presence and/or quantity of the analyte to be detected. The present invention will be described mainly in terms of an immunoassay, wherein the capture reagent and/or detection reagent can comprise, for example, an antibody. The present invention is not, however, limited to antibodies and the capture reagent and/or detection reagent can comprise, for example, a nucleic acid, a nucleic acid binding protein, a receptor, a ligand, a nucleic acid, a complementary nucleic acid, a carbohydrate, a lectin, and the like.

The detection reagent can comprise, for example, a SERS-tag that can produce a detectable Raman signal when illuminated with radiation of the proper wavelength. A SERS-tag can encompass any organic or inorganic atom, molecule, compound or structure known in the art that can be detected by Raman spectroscopy. SERS-tags offer the advantage of producing multiple sharp spectral peaks, allowing a greater number of distinguishable labels to be attached to detection probes.

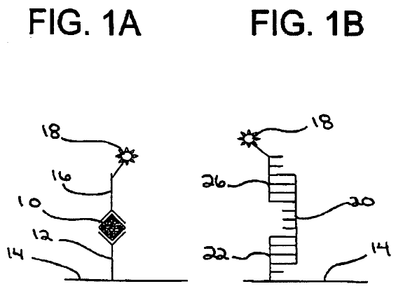

FIGS. 1A and 1B illustrate, in general, sandwich complex formation in protein and nucleic acid detection. Referring to FIG. 1A, a target analyte 10 can be incubated with a binding moiety 12 immobilized to a solid support 14, and with a binding moiety 16 labeled with a signal particle 18. Binding moiety 12 and binding moiety 16 are each capable of specifically binding to target analyte 10 and forming a sandwich complex. Target analyte 10 can comprise, for example, a protein of interest present in a sample, such as blood, serum, urine, or a cervical swab. Binding moieties 12 and 16 each can comprise an antibody, for example a monoclonal antibody, a polyclonal antibody, an antibody fragment, or the like.

For purposes of illustration only, the present invention will be described mainly in terms of an immunoassay wherein target analyte 10 comprises an antigen and each binding moiety comprises an antibody. As used herein, the term “antibody,” is used in its broadest sense to include polyclonal or monoclonal antibodies, as well as antigen-binding fragments of such antibodies. An antibody is characterized, for example, by having specific binding activity for an epitope of an analyte. As such, Fab, F(ab′₂), Fd, and Fv fragments of an antibody that retains specific binding activity for an epitope of an antigen, are included within the definition of an antibody. An antibody includes, for example, naturally-occurring antibodies as well as non-naturally occurring antibodies, including, for example, single chain chimeric, bifunctional, and humanized antibodies. For explanation purposes only, binding moiety 12 immobilized to solid support 14 may be referred to as a “capture antibody”, and binding moiety 16 labeled with signal particle 18 may be referred to a “detection antibody.” The present invention, however, is not limited to embodiments that utilize antibodies, as shown for example in FIG. 1B.

Target analyte 20 can comprise nucleic acid, for example, single-stranded DNA or RNA. Binding moiety 22 can comprise nucleic acid, for example, nucleic acid complementary to at least a portion of target analyte 20. Similarly, binding moiety 26 can comprise nucleic acid, for example, nucleic acid complementary to at least a portion of target analyte 20. Binding moiety 22 can be immobilized to solid support 14, and binding moiety 26 can be labeled with signal particle 18. Binding moiety 22 and binding moiety 26 can each comprise nucleic acid and each can have a length ranging from a single nucleotide to about 100 nucleotides, from about 6 nucleotides to about 50 nucleotides, from about 10 nucleotides to about 25 nucleotide, or about 15 nucleotides. Lengths within or outside of these ranges can be used.

Solid support 14 can comprise a support substrate, for example, a membrane or the interior surface of a sample tube or well. In other embodiments, solid support 14 can comprise a solid particle, for example, a microparticle, a nanoparticle, a bead, or the like. In one or more embodiment, solid support 14 can comprise, for example, a magnetic particle. Magnetic particles and magnetic capture assays are further described in, for example, PCT/US08/57700 filed Mar. 20, 2008 to Natan and entitled “Assays Using Surface-Enhanced Raman Spectroscopy (SERS)-Active Particles)”; U.S. Pat. No. 5,945,281 to Prabhu, which was filed on Feb. 2, 1996 and is entitled “Method and apparatus for determining an analyte from a sample fluid”; U.S. Pat. No. 6,514,415 B1 to Natan, which was filed on Oct. 6, 2000 and is entitled “Surface Enhanced Spectroscopy-active Composite Nanoparticles”; and O. Olsvik, “Magnetic Separation Techniques in Diagnostic Microbiology,” Clinical Microbiology Reviews, Vol. 7, 43-54 (1994), the disclosures of which are hereby incorporated herein by reference in their entirety.

The magnetic particles can comprise, for example, paramagnetic, superparamagnetic, or ferromagnetic materials. The magnetic particles can comprise, for example, paramagnetic microspheres approximately 1 micron in diameter, available from Bangs Laboratories, Inc., Fishers, Ind. The magnetic particles are not limited to this size, and can have a diameter ranging, for example, from about 0.05 micron to about 10.0 microns. The magnetic particles can comprise encapsulated magnetic particles, for example, particles comprising a magnetite-rich core encapsulated with a silica or polymer shell. The magnetic particles can comprise, for example, superparamagnetic material, such as 1 micron to 10 micron beads coated with a material, such as a metal oxide, like silica, available from Bioclone, Inc., San Diego, Calif.

Signal particle 18 can comprise any compound capable of generating a measurable electromagnetic signal, for example a fluorescent, luminescent, calorimetric, and/or Raman signal. The signal particle can also comprise a compound capable of generating a radiolabel, although such compounds are not as compatible in methods and assays that require a wash step. The signal particle can be directly labeled with a signal generating compound, or can be indirectly labeled, for example through a linker or bridge compound. In one or more embodiments, signal particle 18 can comprise, for example, a SERS-active particle, also termed “SERS-labeled nanotag,” a “SERS-nanotag,” or “SERS-tag.” A SERS-tag can produce detectable Raman spectra when illuminated with radiation of the proper wavelength. A SERS-tag encompasses any organic or inorganic atom, molecule, compound, or structure known in the art that can be detected by Raman spectroscopy.

SERS-tags offer the advantage of producing sharp spectral peaks, allowing a large number of distinguishable labels. A number of distinct reporter molecules with strong Raman spectra are known and can be used to create distinct “flavors” of SERS-active particles to enable multiplexing capabilities (the term “flavor” indicates particles that provide distinct Raman signatures upon irradiation). A number of different “flavors” can be excited with a single wavelength. SERS-labeled nanotags are further described, for example, in U.S. Pat. No. 6,514,767 B1 to Natan, which was filed on Oct. 6, 2000 and is entitled “Surface Enhanced Spectroscopy-active Composite Nanoparticles; U.S. Pat. No. 7,192,778 B2 to Natan, which was filed on Jan. 16, 2003 and is entitled “Surface Enhanced Spectroscopy-active Composite Nanoparticles”; and U.S. Patent Application Pub. 2005/0158870 A1 to Natan, which was filed on Feb. 4, 2005 and is entitled “Surface Enhanced Spectroscopy-active Composite Nanoparticles”, and PCT/US08/57700 filed Mar. 20, 2008 to Natan and entitled “Assays Using Surface-Enhanced Raman Spectroscopy (SERS)-Active Particles), the disclosures of which are hereby incorporated herein by reference in their entirety. An example of a SERS-active particle is Nanoplex™ Biotags, available from Oxonica Inc, Mountain View, Calif.

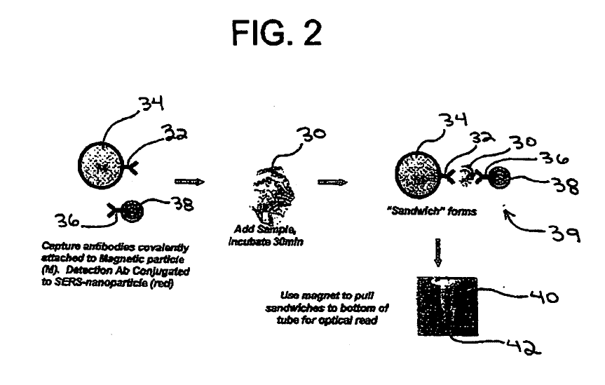

Referring again to FIG. 2, an example of a sandwich immunoassay is illustrated. In particular, FIG. 2 illustrates a no-wash homogeneous immunoassay. In this example, a capture antibody 32 is immobilized to a magnetic particle 34, and a detection antibody 36 is labeled with a signal particle 38. Signal particle 38 can comprise, for example, a SERS-nanoparticle. A sample comprising an antigen 30 is added and the reaction mixture is allowed to incubate. Capture antibody 32 and detection antibody 36 each can specifically bind to a distinct epitope of antigen 30, thus forming a sandwich complex 39.

In the example shown in FIG. 2, the immunoassay binding reaction takes place in a sample tube 40. A magnet (not shown) can then be applied adjacent sample tube 40 to attract sandwich complexes 39 to a pre-defined area of sample tube 40. In one or more embodiments of the present invention, a magnet can be applied adjacent and below sample tube 40 such that a pellet 42, comprising sandwich complexes 39, can be formed in the bottom of sample tube 40. Pellet 42 can then be optically interrogated. Signal particle 38 can generate a signal that corresponds to the presence or absence of sandwich complex 39, and consequently, pellet 42 can be analyzed to determine the concentration of target analyte 30 present in the sample. A sample tube and/or a method of forming a pellet in the sample tube described in PCT/US08/57700, and/or U.S. Published Patent Application No. 2006/0240572 to Carron et al. filed Aug. 24, 2005 entitled “System and method for Raman spectroscopy assay using paramagnetic particles”, herein both incorporated by reference in its entirety, can be utilized in one or more embodiments of the present invention.

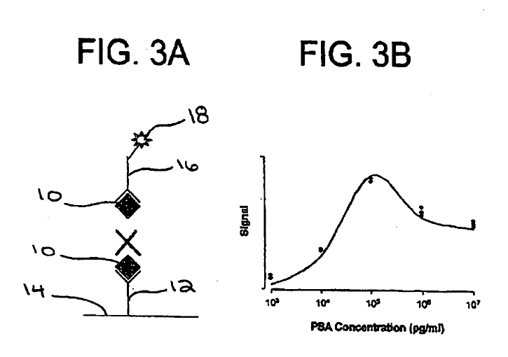

As described above, and as shown in FIGS. 3A and 3B, a homogeneous assay can have limited dynamic range and display a so-called “hook-effect.” Referring to FIG. 3A, the hook-effect occurs when capture antibody 12 and detection antibody 16 bind to separate antigens 10, blocking the formation of a sandwich complex. This phenomenon manifests itself as a drop in signal which is seen in FIG. 3B. Referring to FIG. 3B, the observed signal remains essentially proportional to the concentration of PSA over about 2 orders of magnitude from about 1 ng/ml to about 100 ng/ml. At higher concentrations, however, the signal may actually decrease with increasing antigen level. In FIG. 3B, the hook-effect is seen at concentrations above 100 ng/ml.

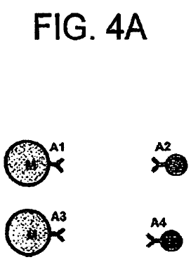

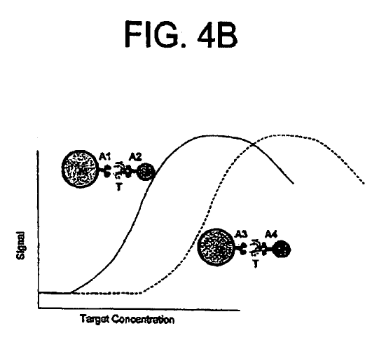

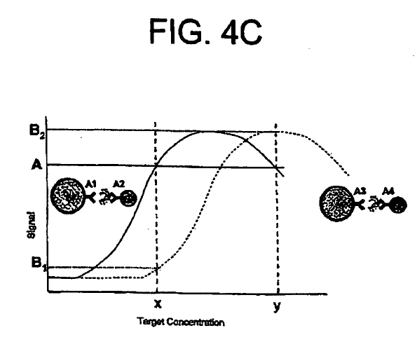

According to certain embodiments, a method for overcoming the hook-effect and expanding the dynamic range of an assay can utilize binding moieties specific for a target analyte that have different affinity for the analyte. The dynamic range can be expanded beyond the hook-effect by at least about one order of magnitude. This is preferred, but not a requirement. The term “order of magnitude,” as used herein, means a factor of ten (10). FIG. 4A illustrates non-limiting examples of binding moieties comprising antibodies.

Referring to FIG. 4A, antibodies A1 and A3 can each specifically bind to a target antigen, and each can have a different affinity for the antigen. A1 can have, for example, a greater affinity for the target antigen than A3. The term “specifically bind,” when used in reference to an antibody means that an interaction of the antibody and a particular epitope has a preferred dissociation constant of less than about 1×10⁻⁶ M. Antibodies A1 and A3 can be immobilized to a solid support, for example to a magnetic particle M (i.e. capture antibody “MA1” and capture antibody “MA3”).

Antibodies A2 and A4 can also each specifically bind to the target antigen and each can also have a different affinity for the antigen. A2 can have, for example, a greater affinity for the target antigen than A4. Antibodies A2 and A4 can each be labeled with a signal particle, S1 and S2 respectively (i.e. detection antibody “A2S1” and detection antibody “A4S2”). S1 and S2 can each be capable of generating a detectable signal that is distinguishable from one another. S1 and S2 can each comprise, for example, a SERS-tag, such as Trans-1,2-Bis(4-pyridyl)-ethylene (BPE), or 4,4′-Dipyridyl (DPY). In preferred embodiments S1 and S2 are SERS-tags, which are detected using Surface Enhanced Raman Spectroscopy.

A target antigen (T) can be incubated with a first pair of capture antibody and detection antibody, for example, MA1 and A2S1, and incubated with a second pair of capture antibody and detection antibody, for example, MA3 and A4S2, under appropriate conditions for forming sandwich complexes such as those described above and shown in FIG. 1A and FIG. 2. Referring to FIG. 4B, a first sandwich complex comprising MA1-T-A2S1 and a second sandwich complex comprising MA3-T-A4S2 can be formed.

Antibodies A1 and A2 can each have a higher affinity for T than antibodies A3 and A4. Accordingly, two different binding profiles can result, as shown in FIG. 4B. The higher affinity antibody pair A1 and A2 can produce a reference profile represented by the solid line. The lower affinity antibody pair A3 and A4 can produce a reference profile represented by the dashed line. Distinguishable signals generated by S1 and S2 can allow the two different binding profiles to be observed.

In one or more embodiments, a standard reference profile for a set of target analyte standards of known concentration can be produced. For the antibody/antigen assay described above, a first standard reference profile can be generated from known amounts of T standard complexed with the same capture antibody and detection antibody pair used to analyze the target antigen. The standard reference profile can resemble, for example, a standard binding curve. The standard binding curve can resemble, for example, the binding profiles shown in FIG. 4B. In some embodiments, at least two standard reference profiles can be generated, one from each antibody pair.

Generally, the concentration of the target analyte can be determined by comparing the first signal and the second signal to the corresponding standard reference profile. The target analyte concentration can be determined, for example, as shown in FIG. 4C.

Referring to FIG. 4C, a first sandwich complex comprising, for example, MA1-T-A2S1, can generate a measured signal indicated by A. Because S1 can generate a signal distinguishable from other signals, signal A can be associated with the first sandwich complex. Signal A, can be compared to a first standard reference profile, and a potential target antigen concentration can be determined. Because of the hook-effect, however, signal A can potentially correspond to two different analyte concentrations, indicated by X and Y. This ambiguous result can be unacceptable in a quantitative application or, for example, in a clinical setting in which determination of X and Y leads to different diagnoses.

To resolve this ambiguity, in the present invention, a second sandwich complex comprising, for example, MA3-T-A4S2, can be incorporated in the assay. The second sandwich complex can generate a second measured signal, for example B₁. Because S2 can generate a distinguishable signal, signal B₁ can be associated with the second sandwich complex. Signal B₁ can be compared to a second standard reference profile, and the target antigen concentration can be absolutely determined as X. Alternatively, the second sandwich complex can generate a second measured signal, for example B₂. The second measured signal B₂ can be again compared to the second standard reference profile and the target antigen concentration can be absolutely determined as Y. The dynamic range of the assay can be expanded beyond target analyte concentrations normally affected by the hook-effect. The dynamic range can be expanded, for example, by at least about one order of magnitude.

In one or more embodiments, a signal intensity ratio, for example a ratio of the first signal and second signal (A/B), can be determined. The signal intensity ratio, A/B, can be compared to a standard reference profile that is based on the ratio of the first signal to the second signal, A/B, for one or more standards of known concentration. In one or more embodiments, for example, the concentration of a target analyte in a sample can be absolutely determined by referencing a signal intensity ratio measured alongside a standard reference profile. As an option, one can use the reference spectrum in the assay instead of a standard curve as mentioned herein.

The present invention does not require two separate binding moiety pairs (e.g., A1-A2, and A3-A4). A single binding moiety pair can be “flipped” wherein each binding moiety can be immobilized or labeled on the opposite particle. The method can utilize a first pair of binding moieties, for example, A1 immobilized to solid support M, and A2 labeled with signal particle S1. A1 and A2 can also be flipped such that A2 can be immobilized to M, and A1 can be labeled with signal particle S2, to provide a second pair of binding moieties. Target analyte T can be incubated with both binding moiety pairs, wherein a first sandwich complex comprising MA1-T-A2S1 and a second sandwich complex comprising MA2-T-A1S2 can be formed. The two “flipped” sandwich complexes can exhibit different binding affinity for T. Accordingly, two different binding profiles can be demonstrated, such as the profiles illustrated in FIG. 4B.

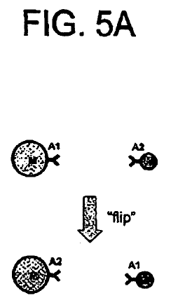

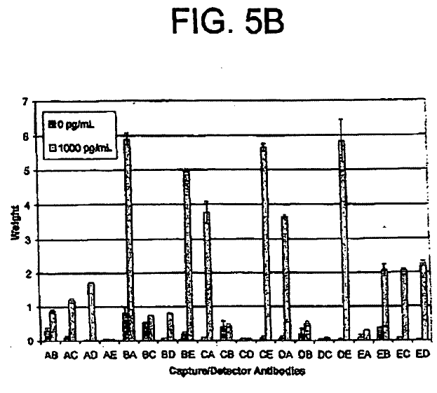

For example, as shown in FIG. 5A, antibodies A1 and A2 can be “flipped,” wherein capture antibody A1 can be labeled with signal particle S2, and detection antibody A2 can immobilized on solid support M. In this example, two antibody pairs can thus be provided, MA1-A2S1 and MA2-A1S2. It has been observed that flipping the antibodies can result in a significant difference in assay sensitivity.

FIG. 5B shows experimental results in which five antibodies (each antibody assigned a letter A-E) specific for Thyroid-Stimulating Hormone (TSH) were immobilized to magnetic particles and labeled with SERS-tags in each possible combination. The antibody pair of each assay is coded by two letters, the first letter corresponding to the antibody immobilized to the magnetic particle and the second letter corresponding to the antibody labeled with the SERS-tag. The signal level of a homogenous no-wash assay at two TSH levels (0 and 1000 pg/ml) was measured.

The bar graph in FIG. 5B shows the flipped antibody combination pair for each assay. For a given antibody pairing, the signals at the two TSH levels (0 and 1000 pg/mL) gives an indication of the assay sensitivity over the concentration range of 0-1000 pg/mL. As can be seen by comparing the signal level at 1000 pg/mL to the signal level at 0 pg/mL for each pairing combination, different assay sensitivities can be obtained when antibody pairing is switched, or “flipped”. In the present invention, the flipped antibody pairs can show enough difference in analyte response to obviate a need for additional antibody pairs, and this is one option to achieve the purposes of the present invention.

In an alternative embodiment, binding moiety pairs having different affinity for a target analyte can be generated by altering the immobilization and/or labeling strategy. For example, a first pair is A1 immobilized to a solid support M, and A2 labeled with a signal particle S1. A second pair is A1 immobilized to a solid support M, and A2 labeled with a signal particle S2. A1 can, however, be immobilized to M using one type of chemistry in the first pair, and can be immobilized to M using a different type of chemistry in the second pair. Similarly, A2 can be labeled with S1 using one type of chemistry, and can be labeled with S2 using a different type of chemistry. The different chemistries generate different affinities of A1 and A2 for a target analyte. For example, some chemistries can orient an antibody so that its binding site is more accessible, while other chemistries can make the binding site less accessible, thereby lowering the affinity.

The present invention does not require that the signals for the reagent pair/analyte sandwich complexes be measured separately and/or independently. One or more signals can be measured simultaneously and a combined signal can be provided. Each signal can be generated by a signal particle associated with each sandwich complex, and each signal is capable of being distinguished from one another. Because the signals are distinguishable, the combined signal can be parsed into the separate signals, which can then be analyzed.

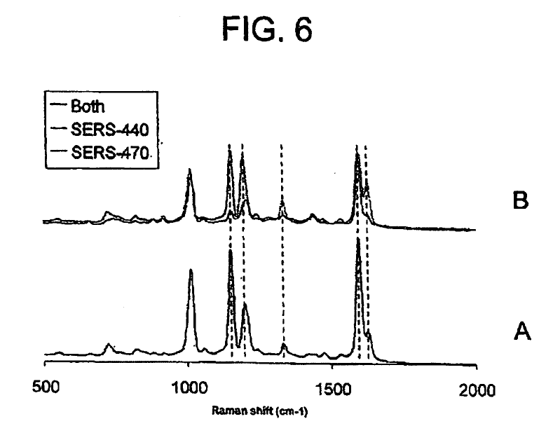

For example, a first signal can be generated from a first sandwich complex and a second signal can be generated from a second sandwich complex, wherein each signal is distinguishable from the other. The first signal and the second signal can be measured to produce a combined signal and then the combined signal can be parsed into separate signals. The separate signals can be parsed wherein they are each capable of being compared to a standard reference profile. The first signal and the second signal can be measured simultaneously. An example of this embodiment is explained further in FIG. 6.

FIG. 6 displays a SERS spectrum produced from a first signal and a second signal generated by two different SERS-tags (Oxonica Nanoplex™ Biotags with reporters Trans-1,2-Bis(4-pyridyl)-ethylene and 1,2-di(4-pyridyl)acetylene). Spectrum A shows a composite spectrum taken from both SERS-tags. Individual peaks from both individual SERS-tags can be observed, as well as combined peaks. As shown in spectrum B, composite spectrum A can be parsed into separate signals from the two individual SERS-tags, and individual Raman spectra of each signal particle can be distinguished.

The present invention is not limited to two binding moiety pairs. In an alternative embodiment, more than two binding moiety pairs can be utilized to determine the concentration of a target analyte. If two binding moiety pairs, for example, do not provide sufficient resolution to unambiguously determine the analyte level in an assay, additional binding pairs can be used. The dynamic range of an assay can be expanded by utilizing more than two binding moiety pairs, wherein each pair can be capable of forming a sandwich complex with the target analyte. Each sandwich complex can have a different affinity for the target analyte, and each sandwich complex can include a signal particle that generates a distinguishable signal. Two or more, such as three to six, or more binding moiety pairs can be utilized.

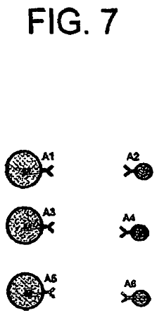

Referring to FIG. 7, three (or more) immunoassay antibody pairs can be provided, A1-A2, A3-A4, and A5-A6. Each antibody pair can comprise a capture antibody immobilized to a solid support and a detection antibody labeled with a signal particle (i.e., MA1/A2S1, MA3/A4S2, and MA5/A6S3). Each antibody can have a different affinity for the target antigen, and each signal particle can generate a distinguishable signal. Each antibody pair can form a sandwich complex with the target analyte, which can produce a distinct binding profile, as shown for example, by the binding profiles in FIG. 4C.

In alternative embodiments, the method and assay can be used for multiplex analysis of a plurality of target analytes. As used herein, the phrase “multiplex” refers to the detection and/or or analysis of more than one target analyte of interest. Multiplex refers to at least 2 different target analytes. In other embodiments at least 3 different target analytes, at least 6 different target analytes, and at least 10 or more different target analytes can be detected and/or analyzed, although the present invention is not limited to the number of target analytes in a multiplex analysis. Multiple sandwich complexes can form with multiple target analytes in the same reaction container.

The method can comprise incubating a plurality of target analytes, for example in a sample, with a plurality of binding moiety pairs under conditions suitable to form sandwich complexes. Each binding moiety pair can comprise a binding moiety labeled with a signal particle that can generate a distinguishable signal. Following sandwich complex formation, the individual signals can be detected in a multiplex manner with a suitable detection device. The individual signals can be analyzed, for example, via a least-squares fitting technique.

Each signal particle can comprise a SERS-tag associated with a unique optical signature. Because each target analyte is bound to a specific detection reagent comprising a known SERS-tag that emits a distinguishable signal, individual signals detected from the sandwich complexes can thus be associated with the identity of the target analyte.

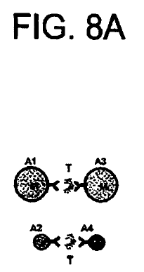

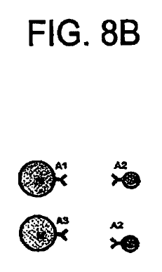

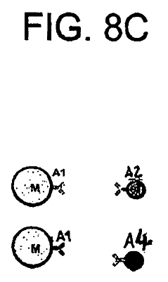

Multiple antibody pairs incorporated together in a no-wash homogeneous assay can lead to the formation of unwanted sandwich complexes. Referring to FIG. 8A, unwanted sandwich complexes can comprise, for example, two capture antibodies (e.g., MA1-T-A3M) which therefore fail to produce a detectable signal, or two detection antibodies (e.g., S1A2-T-A4S2) which can produce a mixed signal or can fail to be separated (e.g., pelleted) from the reaction mixture. Unwanted complexes such as those shown in FIG. 8A can reduce the sensitivity of an assay system.

Embodiments of the present invention specifically address the problem of unwanted complexes. Two or more binding moiety pairs can utilize the same binding moiety immobilized to a solid support. Two or more binding moiety pairs can utilize the same binding moiety labeled with distinguishable signal particles. Examples of these embodiments are explained further in FIG. 8B.

Referring to FIG. 8B, antibody binding pairs can comprise an antibody, A2, labeled with two different signal particles, S1 and S2. Antibody binding pairs A1-A2 and A3-A2 can then be utilized, wherein sandwich complex MA1-T-A2S1 and MA3-T-A2S2 can form. Because detection antibodies A2S1 and A2S2 can be specific for the same epitope on a target antigen, the unwanted two detection antibody complexes such as those shown in FIG. 8A (i.e., A2S1-A2S2 complexes) can not form.

Similarly, and as shown in FIG. 8C, antibody binding pairs can comprise an antibody, A1, immobilized to a solid support, M. Antibody binding pairs A1-A2 and A1-A4 can then be utilized, wherein sandwich complexes MA1-T-A2S1 and MA1-T-A4S2 can form. Because the capture antibody, MA1, can bind to the same epitope on a target antigen in both binding pairs, the unwanted two capture antibody complexes such as those shown in FIG. 8A (i.e., MA1-MA3 complexes) cannot form.

The problem of unwanted complex formation can also be overcome by utilizing a binding moiety that binds to the target analyte to the exclusion of any other binding moiety. For example, a first binding moiety can bind to the target analyte and block or exclude another binding moiety from binding to the target analyte. The first binding moiety can comprise, for example, an antibody, and the other binding moiety can comprise an antibody that binds to the same epitope, or a closely situated epitope, of a target antigen as the first binding moiety.

For example, to illustrate, a pair of capture antibodies, A1 and A3, can be utilized in an assay wherein the antibodies bind to the same epitope on a target antigen. A1 can bind to the target antigen and thus block or exclude further binding by A3. Likewise A3 can bind to the target antigen and block or exclude further binding by A1. In another example, a pair of detection antibodies, A2 and A4, can be similarly used, wherein the antibodies bind to the same epitope. The binding of one detection antibody, for example A2, to the target antigen blocks or excludes binding by the other, in this instance A4, and vice versa. In both examples, the formation of unwanted capture antibody complexes and/or detection antibody complexes can be reduced or eliminated.

The present invention has been described mainly in terms of an immunoassay, but it is not limited in this aspect. Any set of capture/detection binding pairs can be used to detect any analyte of interest. The present invention can be used to detect and quantitate, for example, a nucleic acid. The present invention can be used to detect and quantitate a specific DNA sequence.

The term “nucleic acid” is used broadly herein to mean a sequence of deoxyribonucleotides or ribonucleotides that are linked together by a phosphodiester bond. A nucleic acid can be RNA or can be DNA, which can be a gene or a portion thereof, a cDNA, a synthetic polydeoxyribonucleic acid sequence, or the like, and can be single stranded or double stranded, as well as DNA/RNA hybrid. A nucleic acid can contain nucleoside or nucleotide analogs, or a backbone bond other than a phosphodiester bond.

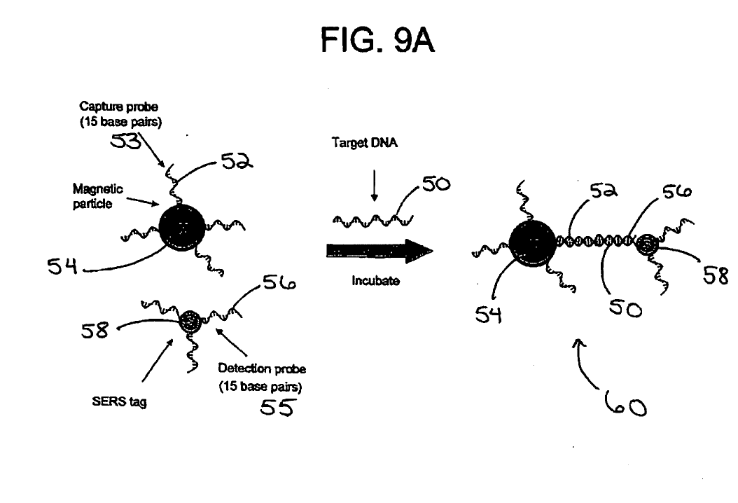

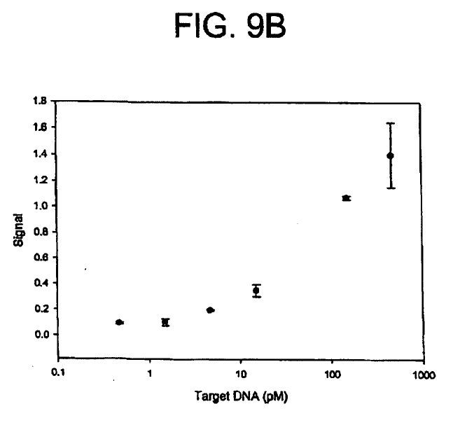

FIG. 9A illustrates an example of a nucleic acid based binding assay, according to one or more embodiments of the present invention. A target analyte 50 can comprise single-stranded DNA and have a sequence, for example, in a range of about 10 nucleotides to about 1000 nucleotides, although the present invention is not limited to a nucleic acid in this size range. A capture reagent 53, can comprise a first binding moiety 52, and a solid support 54. First binding moiety 52 can comprise nucleic acid and have a sequence, for example, in a range of about 10 nucleotides to about 1000 nucleotides, although the present invention is not limited to a nucleic acid in this size range. Binding moiety 52 can be immobilized to solid support 54, such as a magnetic particle. A detection reagent 55 can comprise a second binding moiety 56, and a signal particle 58. Second binding moiety 56 can comprise nucleic acid and have a sequence, for example, in a range of about 10 nucleotides to about 1000 nucleotides, although the present invention is not limited to a nucleic acid in this size range. Second binding moiety 56 can be labeled with signal particle 58, for example a SERS-tag.

First binding moiety 52 and second binding moiety 56 can each be capable of binding to target analyte 50. First binding moiety 52 and second binding moiety 56 can each have a nucleotide sequence that is complementary to target analyte 50. First binding moiety 52 and/or second binding moiety 56 can each have a nucleotide sequence that is, for example, 100 percent complementary to target analyte 50, or a range of about 70 percent to about 100 percent complementary, about 80 percent to about 98 percent complementary, or about 90 percent to about 95 percent complementary to target analyte 50. The skilled person is aware of the degree of complementariness and the hybridization conditions required to cause the target analyte to bind to the first (52) and second (56) binding moieties. The nucleotide sequence of first binding moiety 52 and/or second binding moiety 56 can differ from the nucleotide sequence of target analyte 50 by, for example, one nucleotide, although the present invention does not limit the difference to one nucleotide.

As shown in FIG. 9A, target analyte 50 can be incubated with a first reagent pair comprising first binding moiety 52 immobilized to solid support 54, and second binding moiety 56 labeled with signal particle 58. A sandwich complex 60 can form. Sandwich complex 60 can be capable of generating a signal, for example, Raman scattering spectra, which can be detected. The signal can be analyzed to determine the amount of sandwich complex 60 and thus, the concentration of target analyte.

FIG. 9B illustrates a graph representing a typical binding profile of a nucleic acid-based assay utilizing nucleic acid binding moieties, according to one or more embodiments of the present invention. The graph shows increasing signal with increasing target concentration. FIGS. 9A-B illustrate the sandwich complex and binding profile for one reagent pair. As described in previous embodiments, this embodiment contemplates multiple reagent pairs, each pair having a different binding affinity for the target analyte.

Target analyte 50 can simultaneously be incubated with the first reagent pair and with a second reagent pair comprising a third binding moiety immobilized to a solid support, and a fourth binding moiety labeled with a signal particle, wherein a first sandwich complex and a second sandwich complex can form (not shown).

The first reagent pair can have a different affinity (i.e., higher affinity or lower affinity) for target analyte 50 than the second reagent pair. For example, the third binding moiety and/or the fourth binding moiety can comprise nucleic acid and have a nucleotide sequence that is different than the nucleotide sequence of first binding moiety 52 and/or second binding moiety 56. The different affinity can result, for example, from one or more mismatches included in the sequence of any or all of the binding moieties. For example, at least one of first binding moiety 52, second binding moiety 56, third binding moiety, and/or fourth binding moiety can differ by only a single nucleotide. The single nucleotide difference, for example, can correspond to the location of a single-nucleotide polymorphism (SNP) represented in target analyte 50.

First sandwich complex 60 can be capable of generating a first signal, and the second sandwich complex can be capable of generating a second signal that is distinguishable from the first signal. The first and second signals can be measured and compared to first and second standard reference profiles, respectively.

The present invention also relates to a system for detecting a target analyte (T), the system having an expanded dynamic range. The system can comprise a sample comprising a target analyte, a first reagent pair capable of forming a first sandwich complex with T, and capable of generating a first signal, a second reagent pair capable of forming a second sandwich complex with T, and capable of generating a second signal, wherein the first reagent pair has an affinity for T that is different than the affinity the second pair has for T, an instrument capable of detecting the first signal and the second signal, and an analyzer capable of analyzing the detected first signal and detected second signal. The system can have an expanded dynamic range that reduces a hook-effect, when compared to an assay system that utilizes solely analyzing the detected first signal. The dynamic range can be expanded by at least about one order of magnitude.

The system of the present invention can comprise an instrument capable of providing an excitation light beam. The excitation light beam can be directed toward the sample, and can be capable of inducing the first reagent pair to generate the first signal and/or inducing the second reagent pair to generate the second signal. The instrument can comprise, for example, a laser. The instrument can be capable of inducing Raman scattering spectra. The instrument can be capable of detecting the Raman scattering spectra. The instrument can comprise, for example, a spectrometer. The instrument can simultaneously detect the first signal and the second signal. Examples of an instrument that can be used in one or more embodiments of the present invention are provided in PCT/US08/57700, the disclosure of which is hereby incorporated herein by reference in its entirety.

The system can be utilized to analyze a sample that comprises a homogenous reaction mixture. The homogenous reaction mixture can comprise, for example, the target analyte, the first reagent pair, and the second reagent pair, added and incubated together to form a first and second sandwich complex, wherein no wash steps were performed before analyzing the sample. The first reagent pair and the second reagent pair can be added together to the sample, and the first and second sandwich complexes can simultaneously form.

The system can further comprise a first standard reference profile, and a second standard reference profile. The analyzer can compare the first signal to the first standard reference profile, and compare the second signal to the second standard reference profile. Based upon the comparison, the analyzer can determine, for example, the target analyte concentration. The analyzer can comprise, for example, an information processing unit or computer so that analytical data can be manipulated or stored electronically.

The system can further comprise a sample tube, wherein the sample, the first reagent pair, and the second reagent pair are added together to the sample tube. The system can further comprise a magnet positioned adjacent to the sample tube, wherein the magnet is capable of attracting the first sandwich complex and the second sandwich complex to form a pellet in the bottom or at the side of the tube. Examples of a sample tube, and examples of a system comprising a magnet that can be utilized in the present invention can be found in, for example, PCT/US08/57700, the disclosure of which is hereby incorporated by reference in its entirety.

The present invention also relates to a composition. The composition can comprise a first sandwich complex comprising a target analyte and a first reagent pair having a first affinity for the target analyte, and capable of generating a first signal, and a second sandwich complex comprising the target analyte and a second reagent pair having a second affinity for the target analyte that is different than the first affinity, and capable of generating a second signal. The first signal and the second signal can be distinguishable from each other. The first signal and/or the second signal can comprise Raman spectra.

The first reagent pair and/or the second reagent pair can comprise a first antibody immobilized to a solid support and a second antibody labeled with a signal particle. The solid support can comprise, for example, a magnetic particle. The signal particle can comprise, for example, a SERS-tag. The first affinity and the second affinity can differ by at least about one order of magnitude.

The target analyte, the first reagent pair, and/or the second reagent pair can comprise nucleic acid. In one or more embodiment, at least one of the target analyte, the first reagent pair, and the second reagent pair can comprise a nucleic acid, and at least one of the target analyte, the first reagent pair, and the second reagent pair can comprise a nucleic acid binding protein.

The target analyte can comprise, for example, protein, carbohydrate, nucleic acid, hormone, drug, metabolite, bacteria, fungus, protozoa, cell, and virus. The target analyte can comprise, for example, oncology markers, such as PSA (prostate specific antigen) AFP (alpha fetaprotein), CEA (carcinoembryonic antigen), and p16, human papilloma virus proteins, such as E6 and/or E7 proteins, influenza virus, hormones, such as TSH, and hCG, mini-chromosomal maintenance (MCM) family members, cardiac markers, creatine kinase-subtype MB (CK-MB) and troponin.

The composition can further comprise at least a third sandwich complex. The third sandwich complex can comprise the target analyte and a third reagent pair having a third affinity for the target analyte that is different than the first affinity and the second affinity, and is capable of generating a third signal. The third signal can be distinguishable from the first signal and the second signal.

The composition can comprise at least a second target analyte. The composition can further comprise at least a third sandwich complex and a fourth sandwich complex. A third sandwich complex can comprise a second target analyte and a third reagent pair having a third affinity for the second target analyte, and can be capable of generating a third signal. A fourth sandwich complex can comprise the second target analyte and a fourth reagent pair having a fourth affinity for the second target analyte that is different than the third affinity, and can be capable of generating a fourth signal. The third signal and the fourth signal can be distinguishable from the first signal, the second signal, and from one another.

Applicants specifically incorporate the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

Other embodiments of the present invention will be apparent to those skilled in the art from consideration of the present specification and practice of the present invention disclosed herein. It is intended that the present specification and examples be considered as exemplary only with a true scope and spirit of the invention being indicated by the following claims and equivalents thereof.

What is claimed is:

1. A method for determining the concentration of a target analyte (T) in a sample, comprising:
incubating the sample with a first reagent pair to form, T is present, a first sandwich complex, and with a second reagent pair to form, if T is present, a second sandwich complex, wherein the first reagent pair has a higher affinity for the T than does the second reagent pair;
measuring a first signal generated by the first sandwich complex, and measuring a second signal generated by the second sandwich complex; and
comparing the measured first signal to a first standard reference profile, and comparing the second measured signal to a second standard reference profile.

2. The method of claim 1, wherein the T concentration is determined based on the comparison of the measured first signal to the first reference profile, and the measured second signal to the second reference profile.

3. The method of claim 1, wherein T comprises a protein, carbohydrate, nucleic acid, hormone, drug, metabolite, bacteria, fungus, protozoa, cell, or virus, or any combination thereof.

4. The method of claim 1, wherein the first signal or the second signal or both are generated by a SERS-tag.

5. The method of claim 1, wherein the first reagent pair or the second reagent pair or both comprises an antibody.

6. The method of claim 1, wherein the first reagent pair or the second reagent pair or both comprises nucleic acid.

7. The method of claim 1, wherein the first signal or the second signal or both comprises Raman spectra.

8. The method of claim 1, wherein the binding affinity of the first reagent pair is at least one order of magnitude greater than the binding affinity of the second reagent pair.

9. The method of claim 1, wherein T, the first reagent pair, and the second reagent pair are incubated together simultaneously.

10. The method of claim 1, further comprising incubating the sample, the first reagent pair, and the second reagent pair together in a sample tube;
forming, if T is present, a pellet in a region of the sample tube, the pellet comprising the first sandwich complex and the second sandwich complex; and
measuring the first signal and the second signal generated from the pellet.

11. The method of claim 1, wherein the first signal and the second signal are measured simultaneously.

12. The method of claim 1, further comprising measuring the first signal and the second signal to produce a combined signal and then parsing the combined signal into separate signals capable of being compared to the first and second standard reference profiles.

13. The method of claim 1, wherein the first reagent pair and the second reagent pair are incubated with a sample comprising a plurality of different target analytes.

14. The method of claim 1, further comprising incubating T with one or more additional reagent pair to form one or more additional sandwich complex, wherein the first reagent pair, the second reagent pair, and each of the one or more additional reagent pair have a different affinity for T.

15. The method of claim 1, wherein the first reagent pair comprises single-stranded nucleic acid having a first sequence, and the second reagent pair comprises single-stranded nucleic acid having a second sequence that is different than the first sequence.

16. The method of claim 1, wherein the first reagent pair comprises a first binding moiety (A₁) immobilized to a solid support (M), and a second binding moiety (A₂) labeled with a first signal particle (S₁), and wherein the second reagent pair comprises a third binding moiety (A₃) immobilized to a solid support (M), and a fourth binding moiety (A4) labeled with a second signal particle (S₂) that is distinguishable from S₁.

17. The method of claim 16, wherein A1 is capable of binding to T to the exclusion of A₃, and A₃ is capable of binding to T to the exclusion of A₁.

18. The method of claim 16, wherein A₂ is capable of binding to T to the exclusion of A₄, and A₄ is capable of binding to T to the exclusion of A₂.

19. The method of claim 16, wherein A₁ comprises single-stranded nucleic acid having a first sequence, and A₃ comprises single-stranded nucleic acid having a second sequence that is different than the first sequence.

20. The method of claim 16, wherein A₂ comprises single-stranded nucleic acid having a first sequence, and A₄ comprises single-stranded nucleic acid having a second sequence that is different than the first sequence.

21. The method of claim 16, wherein T comprises single-stranded nucleic acid having a first sequence, and at least one of A₁, A₂, A₃, and A₄ comprises single-stranded nucleic acid having a second sequence that is complementary to the first sequence.

22. The method of claim 21, wherein each of A₁, A₂, A₃, and A₄ comprise single-stranded nucleic acid, and each has a respective sequence that is at least about 80 percent complementary to the first sequence.

23. The method of claim 1,
wherein the first reagent pair comprises a first binding moiety (A₁) and a second binding moiety (A₂), the second reagent pair comprises a third binding moiety (A₃) and a fourth binding moiety (A₄), and wherein A₁ has a greater affinity for T than does A₃.

24. The method of claim 23, wherein A₂ has a greater affinity for T than does A4.

25. The method of claim 23, wherein A₁ has an affinity for T that is at least about an order of magnitude greater than does A₃.

26. The method of claim 1, wherein the first reagent pair comprises a first binding moiety (A₁) immobilized to a solid support (M), and a second binding moiety (A₂) labeled with a first signal particle (S₁), and wherein the second reagent pair comprises A₂ immobilized to a solid support (M), and A₁ labeled with a second signal particle (S₂) that is distinguishable from S₁.

27. The method of claim 1, wherein the first reagent pair comprises a first binding moiety (A₁) immobilized to a solid support (M), and a second binding moiety (A₂) labeled with a first signal particle S₁, and wherein the second reagent pair comprises A₁ immobilized to a solid support (M), and a third binding moiety (A₄) labeled with a second particle (S₂) that is distinguishable from S₁.

28. The method of claim 1, wherein the first reagent pair comprises a first binding moiety (A₁) immobilized to a solid support (M), and a second binding moiety (A₂) labeled with a first signal particle S₁, and wherein the second reagent pair comprises a third binding moiety (A₃) immobilized to a solid support (M), and A₂ labeled with a second signal particle (S₂) that is distinguishable from S₁.

29. The method of claim 1 wherein the method is a homogenous assay.

30. An assay system for detecting a target analyte, the assay system having an expanded dynamic range, comprising;
a sample comprising a target analyte (T);
a first reagent pair capable of forming a first sandwich complex with T, and capable of generating a first signal;
a second reagent pair capable of forming a second sandwich complex with T, and capable of generating a second signal, wherein the first reagent pair has an affinity for T that is different than the affinity the second pair has for T;
an instrument capable of detecting the first signal and the second signal; and
an analyzer capable of analyzing the detected first signal and detected second signal.

31. The assay system of claim 30, having an expanded dynamic range that reduces a hook-effect when compared to an assay system that utilizes solely analyzing the detected first signal.

32. The assay system of claim 30, wherein the dynamic range is expanded at least about one order of magnitude, when compared to an assay system that utilizes solely analyzing the detected first signal.

33. The assay system of claim 30, wherein the instrument is further capable of providing an excitation light beam directed toward the sample, the excitation light beam capable of inducing the first reagent pair to generate the first signal, and capable of inducing the second reagent pair to generate the second signal.

34. The assay system of claim 30, wherein the instrument comprises a laser capable of providing the excitation light beam.

35. The assay system of claim 30, wherein the instrument is capable of detecting Raman scattering spectra generated by at least one of the first signal and the second signal.

36. The assay system of claim 30, wherein the instrument comprises a spectrometer.

37. The assay system of claim 30, wherein the instrument is capable of simultaneously detecting the first signal and the second signal.

38. The assay system of claim 30, wherein the sample further comprises a homogenous assay reaction mixture.

39. The assay system of claim 30, wherein first reagent pair and the second reagent pair are added together to the sample, and the first sandwich complex and the second sandwich complex simultaneously form.

40. The assay system of claim 30, further comprising a first standard reference profile, and a second standard reference profile, wherein the analyzer is capable of comparing the first signal to the first standard reference profile, and of comparing the second signal to the second standard reference profile.

41. The assay system of claim 30, further comprising a sample tube, wherein the sample, the first reagent pair, and the second reagent pair are added together in the sample tube.

42. The assay system of claim 41, further comprising a magnet positioned adjacent to a region of the sample tube, wherein the magnet is capable of attracting the first sandwich complex and the second sandwich complex to form a pellet in the bottom of the tube.

43. The assay system of claim 30 wherein the assay is a homogeneous assay.

44. A composition comprising:
a first sandwich complex comprising a target analyte and a first reagent pair having a first affinity for the target analyte, and capable of generating a first signal; and
a second sandwich complex comprising the target analyte and a second reagent pair having a second affinity for the target analyte that is different than the first affinity, and capable of generating a second signal.

45. The composition of claim 44, wherein each of the first signal and the second signal are distinguishable from the other.

46. The composition of claim 44, wherein at least one of the first signal and the second signal comprises Raman spectra.

47. The composition of claim 44, wherein at least one of the first reagent pair and the second reagent pair comprises a first antibody immobilized to a solid support and a second antibody labeled with a signal particle.

48. The composition of claim 47, wherein the solid support comprises a magnetic particle, and the signal particle comprises a SERS-tag.

49. The composition of claim 44, wherein the first affinity and the second affinity differ by at least about one order of magnitude.

50. The composition of claim 44, wherein at least one of the target analyte, the first reagent pair, and the second reagent pair comprises nucleic acid.

51. The composition of claim 44, wherein the target analyte comprises at least one of protein, carbohydrate, nucleic acid, hormone, drug, cell, metabolite, bacteria, fungus, protozoa, and virus.

52. The composition of claim 44, further comprising at least a third sandwich complex comprising the target analyte and a third reagent pair having a third affinity for the target analyte that is different than the first affinity and the second affinity, and is capable of generating a third signal.

53. The composition of claim 52, wherein the third signal is distinguishable from the first signal and the second signal.

54. The composition of claim 44, further comprising:
a third sandwich complex comprising a second target analyte and a third reagent pair having a third affinity for the second target analyte, and capable of generating a third signal; and
a fourth sandwich complex comprising the second target analyte and a fourth reagent pair having a fourth affinity for the second target analyte that is different than the third affinity, and capable of generating a fourth signal.

55. The composition of claim 54, wherein each of the third signal and the fourth signal are distinguishable from the first signal, the second signal, and one another.
